(19) United States
(12) Patent Application Publication
(10) Pub. No.: US 2023/0190726 A1
(43) Pub. Date: Jun. 22, 2023

(54) COMPOSITIONS AND METHODS FOR TREATING NEGATIVE SYMPTOMS IN NON-SCHIZOPHRENIC PATIENTS

(71) Applicant: Minerva Neurosciences, Inc., Waltham, MA (US)

(72) Inventors: Remy LUTHRINGER, Geneva (CH); Michael DAVIDSON, Tel Aviv (IL)

(21) Appl. No.: 17/825,783

(22) Filed: May 26, 2022

Related U.S. Application Data
(63) Continuation of application No. 16/302,722, filed on Nov. 19, 2018, now abandoned.
(60) Provisional application No. 62/341,590, filed on May 25, 2016.

(51) Int. Cl. A61K 31/454; A61P 25/18

(52) U.S. Cl. CPC A61K 31/454; A61P 25/18

(57) ABSTRACT

The present disclosure describes compositions and methods for treating at least one negative symptom in a human subject who does not have a clinical diagnosis of schizophrenia. The compositions and methods employ a therapeutically effective amount of a compound of formula (I), or a pharmaceutically acceptable salt, hydrate, solvate or polymorph thereof

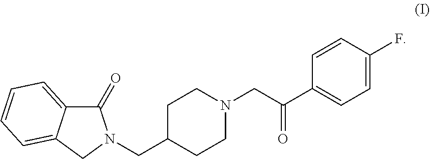 [SMILES: O=C(CN1CCC(CN2Cc3ccccc3C2=O)CC1)c1ccc(F)cc1]

### RELATED APPLICATIONS

This application is a continuation of U.S. application Ser. No. 16/302,722, having a 35 U.S.C. § 371(c) date of Nov. 19, 2018, which is a U.S. National Phase application, filed under 35 U.S.C. § 371(c), of International Application No. PCT/US2017/034030, filed May 23, 2017, which claims priority to, and the benefit of, U.S. Provisional Application No. 62/341,590, filed May 25, 2016, the entire contents of each of which is incorporated herein by reference in their entireties.

### FIELD OF THE DISCLOSURE

The present disclosure in some embodiments relates generally to compositions and methods for treating negative symptoms, and more specifically to treating negative symptoms in patients who do not have a clinical diagnosis of schizophrenia, i.e., non-schizophrenic patients.

### BACKGROUND

Negative symptoms generally refer to a reduction in normal functioning, and include five major sub-domains: blunted affect (affective flattening, blunted expression), alogia (poverty of speech), amotivation (loss of volition), anhedonia (reduced ability to experience or anticipate pleasure) and asociality (social withdrawal). While negative symptoms are a well-documented and intensively studied aspect of schizophrenia, this class of symptoms has been identified in patients with other psychiatric and neurological disorders, including, for example, Alzheimer's disease and other dementias, particularly frontotemporal dementia (FTD), autism spectrum disorder (ASD), bipolar disorder (BPD), major depressive disorder (MDD), Parkinson's disease, temporal lobe epilepsy, stroke, and traumatic brain injury (TBI) (see, e.g., Boone et al, J. of Internat. Neuropsycol. Soc., 2003, Vol 9, pages 698-709; Bastiaansen, J. et al., J. Autsim Dev. Disord. 2011, Vol 41:1256-1266; Getz, K. et al., Am. J. Psychiatry 2002, Vol 159:644-651; Winograd-Gurvich, C. et al., Brain Res. Bulletin, 2006, Vol. 70:312-321; Galynker et al., Neuropsychiatry Neuropsychol Behav Neurol 2000, Vol 13:171-176; Galynker I, et al., J. Nerv. Ment. Dis 1997, Vol 185:616-621; Chaudhury, S., et al., Indian J. of Neurotrauma 2005, Vol 2:13-21; Ameen, S et al., German J. of Psychiatry 2007). Indeed, as early as 2001, it was proposed that negative symptoms are common to mental illnesses generally (Herbener and Harrow, Schizophrenia Bulletin 2001, Vol. 27:527-537). Furthermore, reports of several population studies have concluded that between 20-22% of the general population have one or more negative symptoms, and that the majority of subjects with negative symptoms do not exhibit a clinical diagnosed psychiatric disorder (Werbeloff, N. et al., PLoS ONE 2015, Vol 10:e0119852; Barrantes-Vidal, N., et al., Schizophr. Res. 2010, Vol 122:219-225).

At present, no effective treatments have been approved to treat negative symptoms in schizophrenia or in any other mental disease or neurological condition.

### SUMMARY

The present disclosure is based, in part, on the results of a prospective Phase IIb, 12-week, randomized, double-blind, placebo-controlled parallel clinical trial, which demonstrated a statistically significant benefit of 32 mg and 64 mg doses of MIN-101 over placebo in improving negative symptoms in a cohort of 244 schizophrenic patients with negative symptoms. During this 12-week trial, positive symptoms remained stable and extrapyramidal symptoms (EPS) were absent, consistent with the notion that MIN-101 has a direct and specific effect on negative symptoms rather than improvements on other symptoms. MIN-101 is under clinical development by Minerva Neurosciences (Waltham, Mass.) for the treatment of negative symptoms in schizophrenia.

The active compound in MIN-101 (previously known as CYR-101 and MT-210) has the chemical name 2-{1-[2-(4-Fluorophenyl)-2-oxoethyl]piperidin-4-ylmethyl}-2,3-dihydroisoindol-1-one monohydrochloride dihydrate. The structure of the free base is the compound of formula I (Compound I):

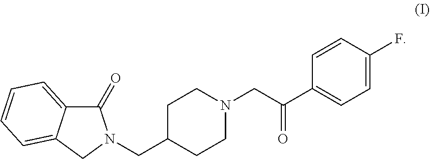 [SMILES: O=C(CN1CCC(CN2Cc3ccccc3C2=O)CC1)c1ccc(F)cc1]

Compound I has specific affinities for sigma₂, 5-hydroxytryptamine-2A (5-HT_{2A}) and at lower affinity levels, α1-adrenergic receptors. MIN-101 exhibits very low or no affinity for other receptors including dopaminergic, muscarinic, cholinergic, and histaminergic receptors. In vivo functional studies have established that MIN-101 is an antagonist at both 5-HT_{2A} and sigma₂ receptors. Two main metabolites of Compound I have been identified and named BFB-520 and BFB-999. The BFB-520 metabolite has been associated with prolongation of QT intervals at supra-therapeutic levels.

In one aspect, the disclosure provides a composition comprising a compound of formula (I) (Compound I), or a pharmaceutically acceptable salt, hydrate, solvate or polymorph thereof, for use in a method of treating at least one negative symptom in a non-schizophrenic human subject, wherein the method which comprises orally administering a therapeutically effective amount of the composition to the subject. In an embodiment, the composition is formulated for oral delivery and the therapeutically effective amount is a total daily dose of Compound I of between about 1 mg to about 64 mg. In an embodiment, the therapeutically effective amount is a total daily dose of Compound I of between about 10 mg to about 64 mg, 20 mg to about 64 mg, or about 30 mg to about 64 mg. In an embodiment, the therapeutically effective amount is a total daily dose of Compound I of about 8 mg, about 16 mg, about 32 mg or about 64 mg.

In another aspect, the disclosure provides a method of treating at least one negative symptom in a non-schizophrenic human subject, wherein the method comprises administering to the subject a therapeutically effective amount of Compound I, or a pharmaceutically acceptable salt, hydrate, solvate or polymorph thereof. In an embodiment, the method comprises orally administering a total daily dose of Compound I of between about 1 mg and about 64 mg. In an embodiment, the total daily dose of Compound I is between about 10 mg to about 64 mg, 20 mg to about 64 mg, or about 30 mg to about 64 mg. In an embodiment, the total daily dose of Compound I is about 8 mg, about 16 mg, about 32 mg or about 64 mg.

In both aspects of the disclosure, the negative symptom to be treated is a primary negative symptom rather than a secondary negative symptom. In an embodiment, the primary negative symptom is selected from the group consisting of: blunted affect, alogia, amotivation, anhedonia and asociality. In an embodiment, the primary negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

In some embodiments of any of the above aspects of the disclosure, the non-schizophrenic patient is diagnosed with a mental disorder or a neurological condition. In an embodiment, the mental disorder or neurological condition is selected from the group consisting of: dementia, frontotemporal dementia (FTD), Alzheimer's disease, autism spectrum disorder (ASD), bipolar disorder (BPD), major depressive disorder (MDD), borderline personality disorder, Parkinson's disease, temporal lobe epilepsy, post-cerebrovascular accident (CVA), traumatic brain injury (TBI), post brain trauma syndrome, mild to moderate mental retardation, viral encephalitis, and drug addiction. In an embodiment, the disorder or condition is FTD or Alzheimer's disease. In an embodiment, the disorder or condition is MDD or BPD. In an embodiment, the disorder or condition is Parkinson's disease.

In some embodiments of any of the above aspects of the disclosure, Compound I or a pharmaceutically acceptable salt, hydrate, solvate or polymorph thereof is administered to the subject for a first treatment period of sufficient length to achieve improvement in at least one negative symptom. In an embodiment, the first treatment period is at least 2 weeks, at least 4 weeks, at least 6 weeks, at least 8 weeks, at least 10 weeks or at least 12 weeks.

In some embodiments of any of the above aspects of the disclosure, if a subject experiences improvement in at least one negative symptom during the first treatment period, then administration of the therapeutically effective dose of Compound I or a pharmaceutically acceptable salt, hydrate, solvate or polymorph thereof is continued for a second treatment period of at least 12 weeks, at least 24 weeks, at least 48 weeks or until the subject is determined to be in remission from the negative symptoms.

In some embodiments of any of the above aspects of the disclosure, Compound I or a pharmaceutically acceptable salt, hydrate, solvate or polymorph thereof is administered in a single dose in the morning or evening. In an embodiment, Compound I or a pharmaceutically acceptable salt, hydrate, solvate or polymorph thereof is administered at least two hours before eating.

In some embodiments of any of the above aspects of the disclosure, a polymorph of Compound I is administered to the subject. In an embodiment, the polymorph is known as Form (A) of Compound (I)·HCl·2H₂O (also referred to herein as Form (A)) and has the characteristics described in the international patent application PCT/US2015/062985 (published as WO 2016/089766) and U.S. patent application Ser. No. 14/954,264 (published as US 2016-0152597 A1), each of which was filed on 30 Nov. 2015, the contents of which are incorporated by reference in their entirety.

In some embodiments of any of the above aspects of the disclosure, Compound I or polymorph Form (A) is administered as part of a pharmaceutical composition which comprises a release modifier that provides a maximum plasma concentration (Cₘₐₓ) of Compound (I) or polymorph Form (A) below 50 ng/mL when a dose of about 1 mg to about 64 mg of the formulation is administered to a human. In an embodiment, the pharmaceutical composition provides a maximum plasma concentration (Cₘₐₓ) for the BFB-520 metabolite of below 10.0 ng/mL, below 5.0 ng/mL, below 4.5 ng/mL, below 4.0 ng/mL, below 3.5 ng/mL, below 3.0 ng/mL, below 2.5 ng/mL, below 2.0 ng/mL, below 1.5 ng/mL, or below 1.0 ng/mL and an area under the curve (AUC) of BFB-520 below 40 hr*ng/mL, below 35 hr*ng/mL, below 30 hr*ng/mL, below 25 hr*ng/mL, below 20 hr*ng/mL, below 15 hr*ng/mL, or below 10 hr*ng/mL.

In some embodiments of any of the above aspects of the disclosure, the human subject is at least 18 years of age, while in other embodiments of any of the above aspects of the disclosure, the human subject is under 18 years of age.

In some embodiments of any of the above aspects of the disclosure, the human subject has not been previously treated with an anti-psychotic drug. In other embodiments of any of the above aspects of the disclosure, the human subject has discontinued prior treatment with an anti-psychotic drug due to experiencing an inadequate response and/or to intolerable side effects

### BRIEF DESCRIPTION OF THE DRAWINGS

The foregoing summary, as well as the following detailed description of the disclosure, will be better understood when read in conjunction with the appended drawings.

FIG. 1 is a graph illustrating the mean change from baseline in the negative subscale score of the Positive and Negative Syndrome Scale (PANSS) of the pentagonal model (Y axis) over 12 weeks of treatment with daily doses of placebo (solid), 32 mg MIN-101 (long dashes) or 64 mg MIN-101 (short dashes).

FIG. 2 is a graph illustrating the mean change from baseline on the PANSS three factors negative symptoms subscale over 12 weeks of treatment with daily doses of placebo (solid), 32 mg MIN-101 (long dashes) or 64 mg MIN-101 (short dashes).

FIG. 3 is a graph illustrating the mean change from baseline in the BNSS total score (Y axis) over 12 weeks of treatment with daily doses of placebo (solid), 32 mg MIN-101 (long dashes) or 64 mg MIN-101 (short dashes).

FIG. 4 is an X-ray powder diffraction of Form (A) of Compound (I)·HCl·2H₂O.

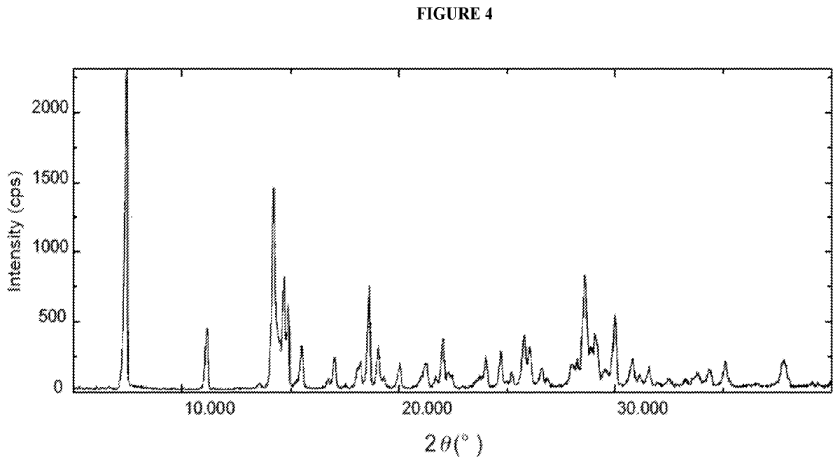

FIG. 5 is an IR spectrum of Form (A) of Compound (I)·HCl·2H₂O.

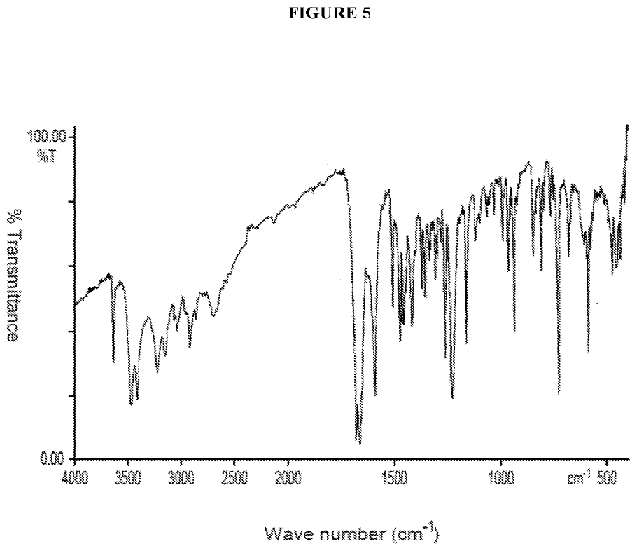

FIG. 6 is a ¹H-NMR spectrum of Form (A) of Compound (I)·HCl·2H₂O.

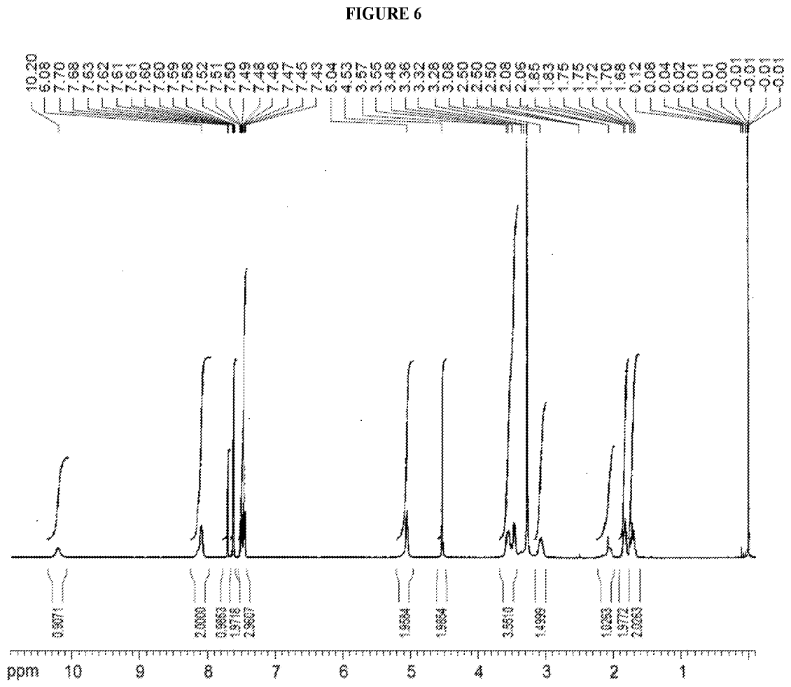

FIG. 7 is a ¹³C-NMR spectrum of Form (A) of Compound (I)·HCl·2H₂O.

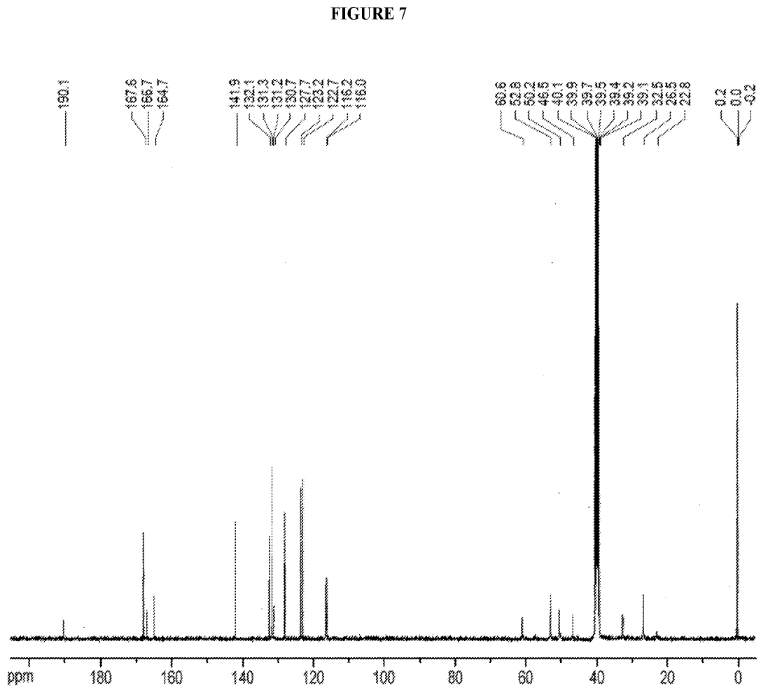

### DETAILED DESCRIPTION

As described in the Examples set forth below, 32 mg and 64 mg daily doses of Compound I have been shown to produce statistically significant improvement in negative symptoms in schizophrenic patients as compared to placebo. Based on these data, and the fact that Compound I antagonizes sigma₂ activity, the present disclosure contemplates that similar improvement in negative symptoms will be achieved in non-schizophrenic human subjects. As used herein, a non-schizophrenic subject means the subject exhibits at least one negative symptom but has not been diagnosed with schizophrenia.

Thus, it is an object of the present disclosure to provide a method of treating at least one negative symptom in a human non-schizophrenic subject comprising administering to the subject a therapeutically effective amount of a composition comprising Compound I or a pharmaceutically acceptable salt, hydrate, solvate or polymorph thereof.

It is also an object of the present disclosure to provide a composition comprising Compound I or a pharmaceutically acceptable salt, hydrate, solvate or polymorph thereof for use in treating at least one negative symptom in a human subject by a method comprising administering to the subject a therapeutically effective amount of the composition.

It is a further object of the present disclosure to employ the compositions and methods of the disclosure to treat at least one negative symptom in a human non-schizophrenic subject who is diagnosed with a mental disorder or neurological condition.

In an embodiment, the negative symptom is one of the five major sub-domains of negative symptoms: blunted affect, alogia, amotivation, anhedonia and asociality. The core characteristics of each sub-domain are described below.

Blunted affect (affective flattening, blunted expression) is characterized by reduced intensity and range of emotional expression as manifested via vocal and non-verbal modes of communication including intonation (prosody), facial expression, hand-gestures and body movements.

Alogia (poverty of speech) is characterized by decreased quantity of speech, reduced spontaneous speech and loss of conversational fluency.

Amotivation (loss of volition) is characterized by deficits in the initiation and maintenance of goal-directed behaviors like work, study, sport, personal hygiene and daily tasks, especially when requiring and effort (cognitive or physical) and significant organization, as well as deficits in desire to undertake such activities. This sub-domain is related to apathy and lack of energy.

Anhedonia (reduced ability to experience or anticipate pleasure) is characterized by the looking forward to a reward, recreational or other pleasurable experience (“wanting”) being more markedly and consistently impaired (anticipatory anhedonia) than the appreciation (“liking”) of the experience itself (consummatory anhedonia).

Asociality (social withdrawal) is characterized by diminished interest in, motivation for, and appreciation of social interactions with others, like family and friends, loss of interest in intimate (sexual) relationships independent of any somatic problems, and for a child, may include loss of interest in playing with other children.

As used herein, unless otherwise noted, the terms “treat”, “treating”, “treatment” and the like, shall include the management and care of a non-schizophrenic subject for the purpose of improving negative symptoms and include administration of Compound I in an amount and for a treatment period that are sufficient to prevent the onset of one or more negative symptoms, reduce the frequency, intensity or severity of one or more negative symptoms, delay or avoid the development of additional negative symptoms, or any combination of these treatment objectives. In an embodiment, the effect of treatment with Compound I is assessed by comparing the severity of the subject's negative symptoms at baseline (e.g., prior to treatment with Compound I) and after at least one treatment period. In an embodiment, the treatment period is at least one week, at least two weeks, at least four weeks, at least six weeks, at least eight weeks, at least 10 weeks or at least twelve weeks.

As used herein, the terms “subject” and “patient” may be used interchangeably, and refer to a human of any age. In an embodiment, the non-schizophrenic subject is six or more years of age. In some embodiments, the subject is at least 18, 19, 20 or 21 years of age. The non-schizophrenic subject exhibits one or more negative symptoms but does not have a diagnosis of schizophrenia. In some embodiments, the non-schizophrenic subject is not diagnosed with a mental disorder or neurological condition. In other embodiments, the non-schizophrenic subject is diagnosed with a mental disorder or neurological condition.

In some embodiments, a composition or method of the disclosure is used to treat a non-schizophrenic subject who is treatment-naive to an anti-psychotic drug. As used herein, an anti-psychotic drug is any drug that does not contain Compound I and has been approved by a regulatory agency for the treatment of psychosis. Examples of atypical antipsychotics include, but are not limited to fluphenazine, risperidone, olanzapine, clozapine, quetiapine, ziprasidone, aripiprazole, sertindole, zotepine, and perospirone.

In other embodiments, a composition or method of the disclosure is used to treat a non-schizophrenic subject who was previously treated with an antipsychotic drug but discontinued such treatment, e.g., because the drug did not provide adequate improvement in the subject's negative symptoms and/or because the subject could not tolerate the side effects of the drug.

In some embodiments, a composition or method of the disclosure is used to treat a non-schizophrenic subject who is treatment-naive to an antidepressant drug. As used herein, an antidepressant drug is any drug that does not contain Compound I and has been approved by a regulatory agency for the treatment of major depressive disorder. Examples of antidepressants include, but are not limited to, fluoxetine, citalopram, escitalopram, venlafaxine, duloxetine, and bupropion.

In other embodiments, a composition or method of the disclosure is used to treat a non-schizophrenic subject who was previously treated with an antidepressant drug but discontinued such treatment, e.g., because the drug did not provide adequate improvement in the subject's negative symptoms and/or because the subject could not tolerate the side effects of the drug.

For purposes of the disclosure encompassed herein, the term “negative symptom” or “negative symptoms” is to be understood as including primary negative symptom(s) typically associated with schizophrenia, the negative symptom(s) measured in the PANSS negative subscale score and the negative symptom(s) measured in the BNSS.

The methods of the disclosure employ administering to the subject a therapeutically effective amount of Compound I, or a pharmaceutically acceptable salt, hydrate, solvate or polymorph thereof. As used herein, the term “therapeutically effective amount” means an amount that is effective to reduce the severity of at least one negative symptom by at least 20%, at least 30%, at least 40%, at least 50%, or at least 60% compared to baseline. Improvement in symptoms in the subject may be measured using any measurement tool generally accepted in the art, including but not limited to the PANSS negative subscale score of the pentagonal model or the Brief Negative Symptom Scale (BNSS) as described herein. In an embodiment, the therapeutically effective amount results in a reduction in the PANSS negative subscale from baseline of ≥20% after 2 weeks, 4 weeks, or 8 weeks of treatment.

In yet another aspect of the disclosure, a composition of the disclosure is formulated and administered to the subject in a manner that provides a dose of Compound I that is substantially equivalent to oral administration of any of the total daily doses specifically described herein. The skilled artisan can readily select formulations and administration routes that would provide such functional equivalence.

The disclosure also provides use of Compound I or a pharmaceutically acceptable salt, hydrate, solvate or polymorph thereof, in the manufacture of a medicament for treating at least one negative symptom in a non-schizophrenic human subject. For example, the medicament is suitable for oral administration. For example, the medicament has a therapeutically effective amount of Compound I, which corresponds to a total daily dose of Compound I of between about 1 mg to about 64 mg.

It will be understood by the skilled artisan that the treating physician may select a dose and dosing regimen within the above guidelines that he or she believes is appropriate based on the health and condition of the subject to be treated, as well as the desired outcome of the treatment. For example, the treating physician may choose to start therapy with a lower than therapeutically effective dose of Compound I and titrate up to a target therapeutically effective dose. For example, the total daily dose of Compound I may be administered in a single dose or in multiple doses.

As used herein, quantitative expressions recited as a range of from about value X to about value Y include any value that is 10% higher or lower than each of X and Y, and also includes any numerical value that falls between X and Y. Thus, for example, a dose of about 32 mg includes a dose of between 30 to 34 mg.

All references to Compound I herein include all pharmaceutically acceptable salts and all solvates and alternative physical forms thereof unless otherwise stated. All doses recited herein are based on the weight of the free base of Compound I, rather than the pharmaceutically acceptable salt, hydrate of solvate thereof or any excipients in the composition, unless otherwise stated. Further, all doses of Compound I recited herein are flat doses (e.g., not dependent on weight of the patient) unless otherwise stated.

For therapeutic administration according to the present disclosure, Compound I may be employed in the form of its free base, but is preferably used in the form of a pharmaceutically acceptable salt. In an embodiment, the form of Compound I used in the compositions and methods of the disclosure is 2-{1-[2-(4-Fluorophenyl)-2-oxoethyl]piperidin-4-ylmethyl}-2,3-dihydroisoindol-1-one monohydrochloride dihydrate, which has a molecular formula of C₂₂H₂₃FN₂O₂, HCl, 2H₂O and a molecular weight of 438.92.

Compound (I) may be synthesized using standard synthetic methods and procedures for the preparation of organic molecules and functional group transformations and manipulations, including the use of protective groups, as can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. A method for preparing Compound (I) is described in U.S. Pat. No. 7,166,617.

The compositions and methods of the disclosure may employ Form (A) of Compound I. Pharmaceutical compositions comprising Form (A) of Compound I may be prepared as described in international patent application PCT/US2015/062985 (published as WO 2016/089766).

In an embodiment, alternative salts of Compound I with pharmaceutically acceptable acids may also be utilized in therapeutic administration, for example salts derived from the functional free base and acids including, but not limited to, palmitic acid, hydrobromic acid, phosphoric acid, acetic acid, fumaric acid, maleic acid, salicylic acid, citric acid, oxalic acid, lactic acid, malic acid, methanesulfonic acid and p-toluene sulfonic acid.

All solvates and all alternative physical forms of Compound I or its pharmaceutically acceptable derivatives as described herein, including but not limited to alternative crystalline forms, amorphous forms and polymorphs, are also within the scope of this disclosure, and all references to a compound of formula I herein (or Compound I) include all pharmaceutically acceptable salts, and all solvates and alternative physical forms thereof.

For therapeutic administration, Compound I or a pharmaceutically acceptable salt thereof, for example, the HCl salt, may be administered in pure form, but will preferably be formulated into any suitable pharmaceutically acceptable and effective composition which provides effective levels of the active ingredient in the body.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an embodiment, the disclosure herein also encompasses pharmaceutically acceptable, hydrates, solvates, stereoisomers, or amorphous solids of the compounds and compositions embodied herein. For example, the term “pharmaceutically acceptable salt” is to describe a salt form of one or more of the compositions herein which are presented to increase the solubility of the compound, for example, in the gastric juices of the patient's gastrointestinal tract in order to promote dissolution and the bioavailability of the compounds and/or compositions. In an embodiment, pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions encompassed by the present disclosure. The term “salt” shall mean any salt consistent with the use of the compounds encompassed by the present disclosure. In the case where the compounds are used in pharmaceutical indications, including the treatment of depression, the term “salt” shall mean a pharmaceutically acceptable salt, consistent with the use of the compounds as pharmaceutical agents.

The term “pharmaceutically acceptable derivative” or “derivative”, as used herein, describes any pharmaceutically acceptable prodrug form (such as an ester or ether or other prodrug group) which, upon administration to a patient, provides directly or indirectly the present compound or an active metabolite of the present compound.

As set forth above, the compositions include pharmaceutically acceptable salts of the compounds in the composition. In other embodiments, the acids which are used to prepare the pharmaceutically acceptable acid addition salts of the aforementioned compounds are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among others.

In an embodiment, compositions comprise base addition salts of the present compounds. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine (meglumine), and the lower alkanolammonium and other base salts of pharmaceutically acceptable organic amines, among others.

As used herein, the term pharmaceutically acceptable salts or complexes refers to salts or complexes (e.g., solvates, polymorphs) that retain the desired biological activity of the parent compound and exhibit minimal, if any, undesired toxicological effects. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

Modifications of a compound can affect the solubility, bioavailability and rate of metabolism of the active species, thus providing control over the delivery of the active species. Further, the modifications can affect the anxiolytic activity of the compound, in some cases increasing the activity over the parent compound. This can easily be assessed by preparing the derivative and testing its activity according to the methods encompassed herein, or other methods known to those skilled in the art.

In an embodiment, the compositions may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions encompassed herein may be administered orally. In other embodiments, compositions may be administered parenterally, by inhalation spray, topically, rectally, nasally, buccally, vaginally or via an implanted reservoir. The term “parenteral” as used herein includes subcutaneous, percutaneous, intravenous, intramuscular, intra-articular, intra-synovial, intrasternal, intrathecal, intrahepatic, intralesional and intracranial injection or infusion techniques. As will be understood by the skilled artisan, in view of the embodiments encompassed herein, the dosage of active ingredient or ingredients (e.g., a compound of formula I) may be adjusted upward or downward based on the selected route of administration. Furthermore, it will be understood that optimizing the dosage of active ingredient for any selected dosage form may be desired and can be achieved by using the methods described herein or known in the art to evaluate the effectiveness of anxiolytic compounds.

The pharmaceutical compositions embodied herein may be orally administered in any orally acceptable dosage form including, but not limited to, capsules, tablets, aqueous suspensions or solutions. In the case of tablets for oral use, carriers which are commonly used include lactose and corn starch. In an embodiment, lubricating agents, such as magnesium stearate, are also added. For oral administration in a capsule form, useful diluents include lactose and/or dried corn starch, as two non-limiting examples. When aqueous suspensions are required for oral use, the active ingredient is combined with emulsifying and suspending agents. If desired, certain sweetening, flavoring or coloring agents may also be added.

The pharmaceutical compositions encompassed by the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In an embodiment, the therapeutically effective amount of Compound I is administered independently of any other medication that is indicated for the treatment of a mental disorder or neurological condition.

In another embodiment, the therapeutically effective amount of Compound I is administered in conjunction with one or more other medications to treat a co-morbid medical condition, including a mental disorder or neurological condition. Such other medications may be administered or co-administered in forms and dosages as known in the art, or in the alternative, as has been described above for administration of compounds of formula I. The other medication(s) may be administered before, after or simultaneously with Compound I during a desired treatment period.

### Exemplary Embodiments

The present disclosure includes, but is not limited to, the following embodiments.

Embodiment 1: a composition comprising a compound of formula I (Compound I);

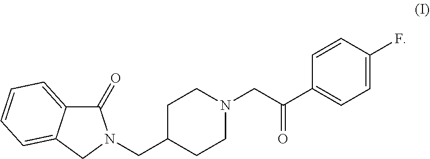 [SMILES: O=C(CN1CCC(CN2Cc3ccccc3C2=O)CC1)c1ccc(F)cc1]

or a pharmaceutically acceptable salt, hydrate, solvate or polymorph thereof, for use in a method for treating at least one negative symptom in a non-schizophrenic human subject, wherein the method comprises orally administering to the subject a therapeutically effective amount of the composition, wherein the therapeutically effective amount is a total daily dose of Compound I of between about 1 mg to about 64 mg.

Embodiment 2: the composition of embodiment 1, wherein the therapeutically effective amount is a total daily dose of Compound I of between about 10 mg to about 64 mg, 20 mg to about 64 mg, or about 30 mg to about 64 mg.

Embodiment 3: the composition of embodiment 2, wherein the therapeutically effective amount is a total daily dose of Compound I of between 30 mg to 64 mg.

Embodiment 4: the composition of embodiment 1, wherein the therapeutically effective amount is a total daily dose of Compound I of about 8 mg, about 16 mg, about 32 mg or about 64 mg.

Embodiment 5: the composition of embodiment 4, wherein the therapeutically effective amount is a total daily dose of Compound I of 32 mg.

Embodiment 6: the composition of embodiment 4, wherein the therapeutically effective amount is a total daily dose of Compound I of 64 mg.

Embodiment 7: a method of treating at least one negative symptom in a non-schizophrenic human subject, wherein the method comprises orally administering to the subject a therapeutically effective amount of Compound I, or a pharmaceutically acceptable salt, hydrate, solvate or polymorph thereof, wherein the therapeutically effective amount is a total daily dose of Compound I of between about 1 mg to about 64 mg.

Embodiment 8: the method of embodiment 7, wherein the total daily dose of Compound I is between about 10 mg to about 64 mg, 20 mg to about 64 mg, or about 30 mg to about 64 mg.

Embodiment 9: the method of embodiment 8, wherein the therapeutically effective amount is a total daily dose of Compound I of between 30 mg to 64 mg.

Embodiment 10: the method of embodiment 7, wherein the total daily dose of Compound I is about 8 mg, about 16 mg, about 32 mg or about 64 mg.

Embodiment 11: the method of embodiment 10, wherein the therapeutically effective amount is a total daily dose of Compound I of 32 mg.

Embodiment 12: the method of embodiment 10, wherein the therapeutically effective amount is a total daily dose of Compound I of 64 mg.

Embodiment 13: use of a compound of formula I (Compound I);

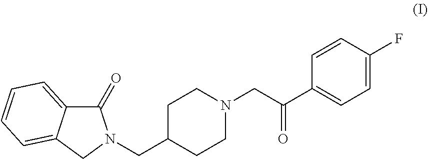 [SMILES: O=C(CN1CCC(CN2Cc3ccccc3C2=O)CC1)c1ccc(F)cc1]

or a pharmaceutically acceptable salt, hydrate, solvate or polymorph thereof, in the manufacture of a medicament for a method for treating at least one negative symptom in a non-schizophrenic human subject, wherein the method comprises orally administering to the subject a therapeutically effective amount of the composition, wherein the therapeutically effective amount is a total daily dose of Compound I of between about 1 mg to about 64 mg.

Embodiment 14: the use of embodiment 13, wherein the therapeutically effective amount is a total daily dose of Compound I of between about 10 mg to about 64 mg, 20 mg to about 64 mg, or about 30 mg to about 64 mg.

Embodiment 15: the use of embodiment 13, wherein the therapeutically effective amount is a total daily dose of Compound I of between 30 mg to 64 mg.

Embodiment 16: the use of embodiment 13, wherein the therapeutically effective amount is a total daily dose of Compound I of about 8 mg, about 16 mg, about 32 mg or about 64 mg.

Embodiment 17: the use of embodiment 16, wherein the therapeutically effective amount is a total daily dose of Compound I of 32 mg.

Embodiment 18: the use of embodiment 16, wherein the therapeutically effective amount is a total daily dose of Compound I of 64 mg.

Embodiment 19: the composition, method, or use of any one of embodiments 1 to 18, wherein the negative symptom is selected from the group consisting of: blunted affect, alogia, amotivation, anhedonia and asociality.

Embodiment 20: the composition, method, or use of any one of embodiments 1 to 18, wherein the negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

Embodiment 21: the composition, method, or use of any one of embodiments 1 to 20, wherein the non-schizophrenic patient is diagnosed with a mental disorder or a neurological condition.

Embodiment 22: the composition, method, or use of embodiment 21, wherein the mental disorder or neurological condition is selected from the group consisting of: dementia, frontotemporal dementia (FTD), Alzheimer's disease, autism spectrum disorder (ASD), bipolar disorder (BPD), major depressive disorder (MDD), Parkinson's disease, temporal lobe epilepsy, post-cerebrovascular accident (CVA), traumatic brain injury (TBI), post brain trauma syndrome, mild to moderate mental retardation, viral encephalitis, and drug addiction.

Embodiment 23: the composition, method, or use of embodiment 22, wherein the mental disorder or neurological condition is FTD or Alzheimer's disease.

Embodiment 24: the composition, method, or use of embodiment 22, wherein the mental disorder or neurological condition is MDD or BPD.

Embodiment 25: the composition, method, or use of embodiment 22, wherein the mental disorder or neurological condition is Parkinson's disease.

Embodiment 26: the composition, method, or use of any one of embodiments 1 to 25, wherein Compound I is administered to the subject for a first treatment period of at least 2 weeks, at least 4 weeks, at least 6 weeks, at least 8 weeks, at least 10 weeks or at least 12 weeks.

Embodiment 27: the composition, method, or use of embodiment 26, wherein, if a subject experiences improvement in at least one negative symptom during the first treatment period, then administration of the therapeutically effective amount of Compound I is continued for a second treatment period of at least 12 weeks, at least 24 weeks, at least 48 weeks or until the subject is determined to be in remission from the negative symptoms.

Embodiment 28: the composition, method, or use of any one of embodiments 1-27, wherein Compound I is administered in a single dose in the morning in fasting condition and at least two hours before eating.

Embodiment 29: the composition, method, or use of any one of embodiments 1 to 28, wherein the polymorph Form (A) of Compound I is administered to the subject.

Embodiment 30: the composition, method, or use of any one of embodiments 1 to 29, wherein Compound I or the polymorph Form (A) of Compound I is administered as part of a pharmaceutical composition which comprises a release modifier that provides a maximum plasma concentration (Cₘₐₓ) of Compound (I) below 50 ng/mL when a dose of about 1 mg to about 64 mg of the formulation is administered to a human.

Embodiment 31: the composition, method, or use of any one of embodiments 29 to 30, wherein the pharmaceutical composition provides a maximum plasma concentration (Cₘₐₓ) for the BFB-520 metabolite of below 5.0 ng/mL, below 4.5 ng/mL, below 4.0 ng/mL, below 3.5 ng/mL, below 3.0 ng/mL, below 2.5 ng/mL, below 2.0 ng/mL, below 1.5 ng/mL, or below 1.0 ng/mL and an area under the curve (AUC) of BFB-520 below 40 hr*ng/mL, below 35 hr*ng/mL, below 30 hr*ng/mL, below 25 hr*ng/mL, below 20 hr*ng/mL, below 15 hr*ng/mL, or below 10 hr*ng/mL.

Embodiment 32: the composition, method, or use of any one of embodiments 1 to 31, wherein the non-schizophrenic subject has not been previously treated with an anti-psychotic drug.

Embodiment 33: the composition, method, or use of any one of embodiments 1 to 31, wherein the non-schizophrenic subject has discontinued prior treatment with an anti-psychotic drug due to experiencing an inadequate response and/or to intolerable side effects.

Embodiment 34: the composition, method, or use of any one of embodiments 1 to 33, wherein the non-schizophrenic subject has not been previously treated with an anti-depressant drug.

Embodiment 35: the composition, method, or use of any one of embodiments 1 to 33, wherein the non-schizophrenic subject has discontinued prior treatment with an anti-depressant drug due to experiencing an inadequate response and/or to intolerable side effects.

Embodiment 36: the composition, method, or use of any one of embodiments 1 to 35, wherein the form of Compound I administered is 2-{1-[2-(4-Fluorophenyl)-2-oxoethyl]piperidin-4-ylmethyl}-2,3-dihydroisoindol-1-one monohydrochloride dihydrate.

Embodiment 37: the composition, method, or use of any one of embodiments 1 to 36, wherein the total daily dose of Compound I is administered in a single dose.

Embodiment 38: the composition, method, or use of any one of embodiments 1 to 36, wherein the total daily dose of Compound I is administered in multiple doses, e.g., twice daily or three or four times daily.

### Positive and Negative Syndrome Scale (PANSS)

Below is a description of the Positive and Negative Syndrome Scale (PANSS) used in the clinical study described in the Examples.

The embodiments encompassed herein are now described with reference to the following Examples. These Examples are provided for the purpose of illustration only and the disclosure encompassed herein should in no way be construed as being limited to these Examples, but rather should be construed to encompass any and all variations which become evident as a result of the teachings provided herein.

### EXAMPLES

### Example 1

### MIN-101 Improves Negative Symptoms in Schizophrenic Patients with Negative Symptoms

A prospective Phase IIb, 12-week, randomized, double-blind, placebo-controlled parallel clinical trial was conducted to evaluate the efficacy, safety and tolerability of MIN-101 in patients with negative symptoms of schizophrenia. The study was designed to evaluate the efficacy of MIN-101 monotherapy on negative symptoms using the pentagonal structure model (PSM) of the Positive and Negative Syndrome Scale (PANSS) as the primary endpoint. A total of 244 patients were randomized in equal groups to receive daily doses of MIN-101 32 mg, MIN-101 64 mg or placebo at 32 clinical sites in Russia and five European countries.

To participate in the trial, patients were required to have stable positive and negative symptoms for three months prior to entry, a PANSS negative sub-score greater than or equal to 20, and scores <4 on the following PANSS items: excitement, hyperactivity, hostility, suspiciousness, uncooperativeness and poor impulse control. The full inclusion and exclusion criteria set forth in the protocol are listed below.

### Inclusion Criteria

Each potential patient must satisfy all of the following criteria before study drug administration to be enrolled in the study:
1. Patient or patient's legal representative has provided informed consent.
2. Male or female patient, 18 to 60 years of age, inclusive.
3. Patient meets the diagnostic criteria for schizophrenia as defined in the Diagnostic and Statistical Manual of Mental Disorders-Fifth Edition (DSM-5), as established by a full psychiatric interview in conjunction with the Mini International Neuropsychiatric Interview (MINI).
4. Patient is stable in terms of positive symptoms of schizophrenia over the last 3 months according to his or her treating psychiatrist.
5. Patient presents with negative symptoms of schizophrenia over the last 3 months according to his or her treating psychiatrist.
6. Patient with PANSS negative subscore of at least 20.
7. Patient with PANSS item score of <4 on:
P4 Excitement, hyperactivity
P7 Hostility
P6 Suspiciousness
G8 Uncooperativeness
G14 Poor impulse control
8. Patients can be on any psychotropic as long as the psychotropic can be discontinued at the beginning of the washout phase without endangering the patient's safety.
9. No change in psychotropic medication during the last month (changes are allowed if done for administrative reasons or with the permission of the Sponsor's Responsible Medical Officer).
10. No history of violence against self, others, or property.
11. Patient in whom, in the opinion of the investigator, a switch to another antipsychotic medication or initiation of an antipsychotic medication is indicated.
12. Female patient, if of childbearing potential, must test negative for pregnancy and must be using a double barrier contraceptive method.
13. Patient must be extensive metabolizers for P450 CYP2D6, as determined by genotyping test before the first drug dose is administered.
14. Patient must be able to understand the nature of the study.
15. The patient is considered by the investigator to be reliable and likely to cooperate with the assessment procedures.

### Exclusion Criteria

Any potential patient who meets any of the following criteria before study drug administration will be excluded from participating in the study:
1. Current bipolar disorder, panic disorder, obsessive compulsive disorder, or evidence of mental retardation.
2. Patient's condition is due to direct physiological effects of a substance (e.g., a drug of abuse, or medication) or a general medical condition.
3. Significant risk of suicide or attempted suicide, or of danger to self or others.
4. Patient has a history of substance abuse within 3 months of the Screening visit (excluding caffeine and cigarette smoking).
5. Positive urine drug screen except when related to prescribed benzodiazepines and opiates recently prescribed for an episode of acute pain (e.g., dental extraction).
6. Patient who cannot be discontinued from psychotropics other than those allowed.
7. Patient who received clozapine within 6 months of the Screening visit. [Country-specific exception: For patients in Russia, a dose of <100 mg/day for the treatment of insomnia is allowed.]
8. Patient receiving treatment with depot antipsychotic medication can be enrolled in the study 4 weeks after the last injection.
9. Patient with a history of significant other major or unstable neurological, neurosurgical (e.g., head trauma), metabolic, hepatic, renal, hematological, pulmonary, cardiovascular, metabolic, gastrointestinal, or urological disorder.
10. Patient with a history of epilepsy seizure disorder (patient with a history of childhood febrile seizure may be enrolled in this study).
11. Patient who has had electroconvulsive therapy (ECT), vagal nerve stimulation (VNS), or repetitive trans-cranial magnetic stimulation (r-TMS) within the 3 months prior to the Screening visit or who are scheduled for ECT, VNS, or r-TMS at any time during the study.
12. Patient with clinically significant abnormalities in hematology, blood chemistry, ECG, or physical examination not resolved by the Baseline visit.
13. Body Mass Index (BMI)>35.
14. Current systemic infection (e.g., Hepatitis B virus [HBV], Hepatitis C virus [HCV], human immunodeficiency virus [HIV], tuberculosis [TB]). Patients with positive Hepatitis B core antibody test and negative Hepatitis B Surface Antigen (HBsAg) may be included in the study if aminotransferase levels (alanine aminotransferase/serum glutamic pyruvic transaminase (ALT/SGPT) and aspartate aminotransferase/serum glutamic oxaloacetic transaminase (AST/SGOT) do not exceed 2 times upper limit of normal (ULN).
15. Patient who requires or may require concomitant treatment with any other medication likely to increase QT interval (e.g., paroxetine, fluoxetine, duloxetine, amiodarone).
16. Patient who requires medication inhibiting the CYP2D6.
17. Patient with a clinically significant electrocardiogram (ECG) abnormality that could be a safety issue in the study, including QT interval value corrected for heart rate using the Fridericia's formula (QTcF)>430 msec for males and >450 msec for females.
18. Patient with a history of myocardial infarction based on medical history or ECG findings at Screening.
19. Familial or personal history of long QT syndrome or with additional risk factors for torsade de Pointes (e.g., hypokalemia, hypomagnesemia).
20. Woman of child-bearing potential, or man, who are unwilling or unable to use accepted methods of birth control.
21. Woman with a positive pregnancy test, is lactating, or is planning to become pregnant during the study.
22. Patient who participated in another clinical study within 3 months prior to Screening.

All three cohorts were balanced with respect to demographic and baseline disease characteristics as shown in the Table immediately below.

| Demographic/Baseline | | MIN-101 | | | |
|---|---|---|---|---|---|
| Characteristics | Placebo | 32 mg | 64 mg | Total | Overall |
| Statistic/Category | (N = 83) | (N = 78) | (N = 83) | (N = 161) | (N = 244) |
| Age (years) | | | | | |
| n | 83 | 78 | 83 | 161 | 244 |
| Mean | 40.0 | 39.8 | 40.6 | 40.2 | 40.2 |
| SD (SE) | 10.2 (1.1) | 10.2 (1.2) | 10.6 (1.2) | 10.4 (0.8) | 10.3 (0.7) |
| Median | 40.6 | 40.8 | 42.0 | 41.0 | 41.0 |
| Min, Max | 21, 59.7 | 18, 59.3 | 19.5, 59.7 | 18, 59.7 | 18, 59.7 |
| Sex | | | | | |
| Male | 48 (57.8%) | 41 (52.6%) | 48 (57.8%) | 89 (55.3%) | 137 (56.1%) |
| Female | 35 (42.2%) | 37 (47.4%) | 35 (42.2%) | 72 (44.7%) | 107 (43.9%) |
| Race | | | | | |
| Caucasian | 83 (100.0%) | 78 (100.0%) | 83 (100.0%) | 161 (100.0%) | 244 (100.0%) |
| Other | 0 (0.0%) | 0 (0.0%) | 0 (0.0%) | 0 (0.0%) | 0 (0.0%) |
| Height (cm) | | | | | |
| n | 83 | 78 | 83 | 161 | 244 |
| Mean | 172.5 | 170.9 | 171.5 | 171.3 | 171.7 |
| SD (SE) | 8.6 (0.9) | 9.7 (1.1) | 8.2 (0.9) | 8.9 (0.7) | 8.8 (0.6) |
| Median | 172.0 | 170.0 | 170.0 | 170.0 | 171.0 |
| Min, Max | 153, 190 | 150, 205 | 156, 196 | 150, 205 | 150, 205 |
| Weight (kg) | | | | | |
| n | 83 | 78 | 83 | 161 | 244 |
| Mean | 77.42 | 74.16 | 75.25 | 74.72 | 75.64 |
| SD (SE) | 14.21 (1.56) | 16.60 (1.88) | 13.70 (1.50) | 15.14 (1.19) | 14.86 (0.95) |
| Median | 78.00 | 73.40 | 75.00 | 74.00 | 75.20 |
| Min, Max | 45.8, 107.5 | 45, 145 | 47.8, 120.5 | 45, 145 | 45, 145 |
| BMI (kg/m\hat{ }2) | | | | | |
| n | 83 | 78 | 83 | 161 | 244 |
| Mean | 26.0389 | 25.2967 | 25.5814 | 25.4435 | 25.6460 |
| SD (SE) | 4.4749 (0.4912) | 4.4992 (0.5094) | 4.3349 (0.4758) | 4.4037 (0.3471) | 4.4279 (0.2835) |
| Median | 26.1710 | 24.9965 | 25.1590 | 25.1590 | 25.3760 |
| Min, Max | 17.856, 37.341 | 16.366, 34.938 | 17.414, 35.062 | 16.366, 35.062 | 16.366, 37.341 |

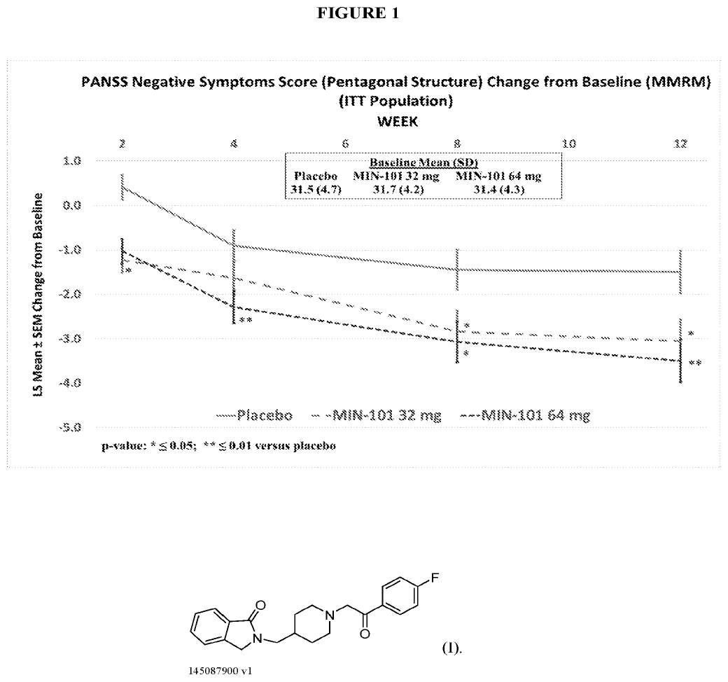

The mean changes from baseline in the PANNS Negative Subscale score in the placebo and treatment arms over 12 weeks of treatment is shown in FIG. 1. A statistically significant improvement was shown for both doses tested: 32 mg: p≤0.023 with an effect size of 0.45, and 64 mg: p≤0.003 with effect size of 0.57.

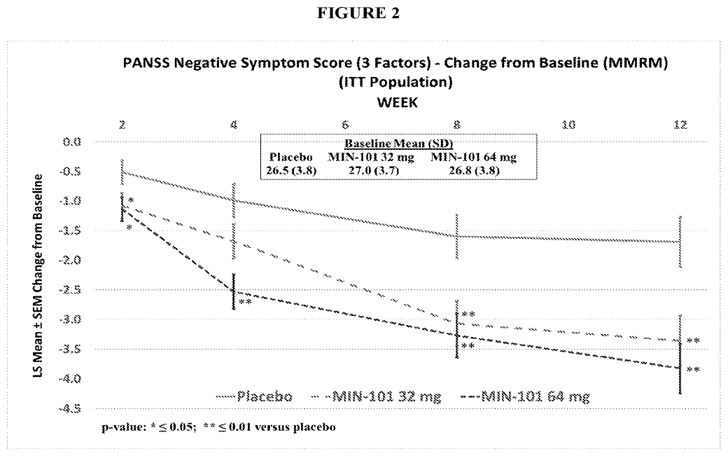

As illustrated in FIG. 2, the study also demonstrated a statistically significant benefit of MIN-101 over placebo on the PANSS three factors negative symptoms subscale for both doses tested: 32 mg: p≤0.006, with an effect size of 0.55, 64 mg: p≤0.001 with an effect size 0.70.

Furthermore, the statistically significant benefit of MIN-101 over placebo was also demonstrated on the PANSS total score (not significant for the 32 mg dose; p≤0.003 for the 64 mg dose), with effect sizes of 0.35 and 0.59, respectively.

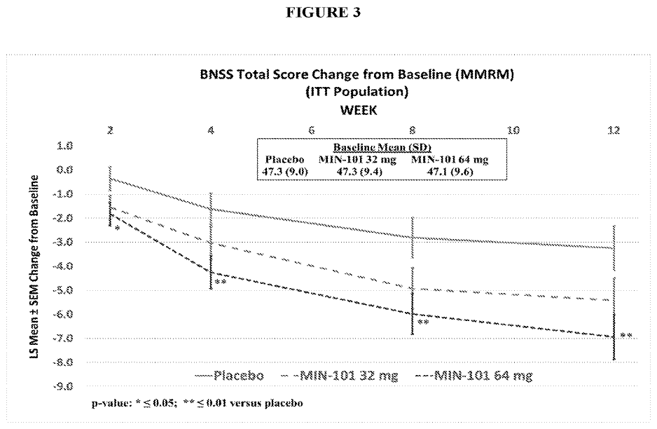

Improvement in negative symptoms achieved by both doses of MIN-101 was also observed when the effect was measured using the BNSS total score, as shown in FIG. 3.

MIN-101 was generally reported to be well tolerated, and the incidence and types of side effects did not differ significantly between the MIN-101 group and the placebo group. Based upon previous non-clinical and clinical experience, QTcF, a measurement of cardiac function, was closely monitored. Discontinuation criteria based on QTcF prolongation were incorporated in the protocol. Two patients out of 162 who received MIN-101 were discontinued based upon these criteria; both of these patients received the higher dose (64 mg). Unlike many currently marketed antipsychotic drugs, no metabolic adverse effects, no weight gain, no extra-pyramidal symptoms and no prolactin elevation were observed.

### EQUIVALENTS AND INCORPORATION BY REFERENCE

The invention has been described herein by reference to certain preferred embodiments. However, as particular variations thereon will become apparent to those skilled in the art, based on the disclosure set forth herein, the invention is not to be considered as limited thereto.

Unless otherwise defined, all technical and scientific terms used herein have the same meaning as commonly understood by one of ordinary skill in the art to which the invention belongs. In the specification and claims, the singular forms also include the plural unless the context clearly dictates otherwise.

It is to be understood that at least some of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present disclosure should not be limited to the performance of their steps in the order written, and one skilled in the art can readily appreciate that the steps may be varied and still remain within the spirit and scope of the present disclosure.

All patents, patent applications, references and publications cited herein are fully and completely incorporated by reference as if set forth in their entirety.

1. A composition comprising a compound of formula I (Compound I): 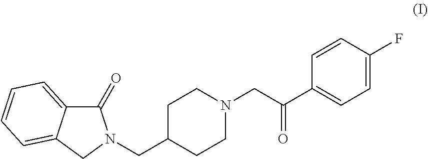 [SMILES: O=C(CN1CCC(CN2Cc3ccccc3C2=O)CC1)c1ccc(F)cc1]
or a pharmaceutically acceptable salt, hydrate, solvate or polymorph thereof, for use in a method for treating at least one negative symptom in a non-schizophrenic human subject, wherein the method comprises orally administering to the subject a therapeutically effective amount of the composition, wherein the therapeutically effective amount is a total daily dose of Compound I of between about 1 mg to about 64 mg.

2. The composition of claim 1, wherein the therapeutically effective amount is a total daily dose of Compound I of between about 10 mg to about 64 mg, 20 mg to about 64 mg, or about 30 mg to about 64 mg.

3. The composition of claim 1, wherein the therapeutically effective amount is a total daily dose of Compound I of 32 mg or 64 mg.

4. The composition of claim 1, wherein the negative symptom is selected from the group consisting of: blunted affect, alogia, amotivation, anhedonia and asociality.

5. The composition of claim 1, wherein the negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

6. The composition of claim 1, wherein the non-schizophrenic patient is diagnosed with a mental disorder or a neurological condition.

7. The composition of claim 6, wherein the mental disorder or neurological condition is selected from the group consisting of: dementia, frontotemporal dementia (FTD), Alzheimer's disease, autism spectrum disorder (ASD), bipolar disorder (BPD), major depressive disorder (MDD), Parkinson's disease, temporal lobe epilepsy, post-cerebrovascular accident (CVA), traumatic brain injury (TBI), post brain trauma syndrome, mild to moderate mental retardation, viral encephalitis, and drug addiction.

8. The composition of claim 7, wherein the mental disorder or neurological condition is FTD, Alzheimer's disease, MDD, BPD or Parkinson's disease.

9. The composition of claim 1, wherein Compound I is administered to the subject for a first treatment period of at least 2 weeks, at least 4 weeks, at least 6 weeks, at least 8 weeks, at least 10 weeks or at least 12 weeks and, if the subject experiences improvement in at least one negative symptom during the first treatment period, then administration of the therapeutically effective amount of Compound I is continued for a second treatment period of at least 12 weeks, at least 24 weeks, at least 48 weeks or until the subject is determined to be in remission from the negative symptoms.

10. The composition of claim 1, wherein the non-schizophrenic subject has not been previously treated with an anti-depressant drug or has discontinued prior treatment with an anti-depressant drug due to experiencing an inadequate response and/or to intolerable side effects.

11. A method of treating at least one negative symptom in a non-schizophrenic human subject, wherein the method comprises orally administering to the subject a therapeutically effective amount of Compound I: 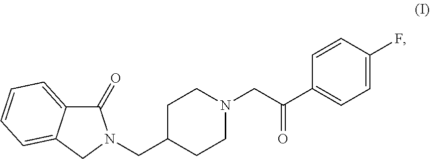 [SMILES: O=C(CN1CCC(CN2Cc3ccccc3C2=O)CC1)c1ccc(F)cc1]
or a pharmaceutically acceptable salt, hydrate, solvate or polymorph thereof, wherein the therapeutically effective amount is a total daily dose of Compound I of between about 1 mg to about 64 mg.

12. The method of claim 11, wherein the total daily dose of Compound I is between about 10 mg to about 64 mg, 20 mg to about 64 mg, or about 30 mg to about 64 mg.

13. The method of claim 11, wherein the total daily dose of Compound I is 32 mg or 64 mg.

14. The method of claim 11, wherein the negative symptom is selected from the group consisting of: blunted affect, alogia, amotivation, anhedonia and asociality.

15. The method of claim 11, wherein the negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

16. The method of claim 11, wherein the non-schizophrenic patient is diagnosed with a mental disorder or a neurological condition.

17. The method of claim 16, wherein the mental disorder or neurological condition is selected from the group consisting of: dementia, frontotemporal dementia (FTD), Alzheimer's disease, autism spectrum disorder (ASD), bipolar disorder (BPD), major depressive disorder (MDD), Parkinson's disease, temporal lobe epilepsy, post-cerebrovascular accident (CVA), traumatic brain injury (TBI), post brain trauma syndrome, mild to moderate mental retardation, viral encephalitis, and drug addiction.

18. The method of claim 17, wherein the mental disorder or neurological condition is FTD, Alzheimer's disease, MDD, BPD or Parkinson's disease.

19. The method of claim 11, wherein Compound I is administered to the subject for a first treatment period of at least 2 weeks, at least 4 weeks, at least 6 weeks, at least 8 weeks, at least 10 weeks or at least 12 weeks and, if the subject experiences improvement in at least one negative symptom during the first treatment period, then administration of the therapeutically effective amount of Compound I is continued for a second treatment period of at least 12 weeks, at least 24 weeks, at least 48 weeks or until the subject is determined to be in remission from the negative symptoms.

20. The method of claim 11, wherein the non-schizophrenic subject has not been previously treated with an anti-depressant drug or has discontinued prior treatment with an anti-depressant drug due to experiencing an inadequate response and/or to intolerable side effects.
